SEQUENCE LISTING (10 sequences)

SEQ ID NO: 1 - protein, 113 aa, synthetic construct
  source 1..113: note = nanobody CSFV-E0-Nb1
ESGGGLVQPGGSLRLSCAGSGIIFSSVTMAWYRQAPGKQREVVARFSSGGRATYADSVEGRFTISRDNVKNMVYLQMNSLAPEDTAVYYCNANWWYERNYDYWGQGTQVTVSS

SEQ ID NO: 2 - DNA, 341 bp, synthetic construct
  source 1..341: mol_type = other DNA; note = Gene encoding CSFV-E0-Nb1
gagtctgggggaggcttggtgcagcctggggggtctctgagactctcctgtgcaggctcaggaataatcttcagtagcgttaccatggcctggtaccgccaggctccagggaagcagcgcgaagtggtcgctcgttttagtagtggtggtcgcgcgacctacgcagactccgtggagggccgattcaccatctccagagacaacgtcaagaatatggtctatctacaaatgaacagcctggcacctgaggacacggccgtctattactgtaatgcgaactggtggtacgagaggaattatgattactggggccaggggacccaggtcaccgtctcctcagc

SEQ ID NO: 3 - DNA, 23 bp, synthetic construct
  source 1..23: mol_type = other DNA; note = Primer F1
gtcctggctgctcttctacaagg

SEQ ID NO: 4 - DNA, 23 bp, synthetic construct
  source 1..23: mol_type = other DNA; note = Primer R1
ggtacgtgctgttgaactgttcc

SEQ ID NO: 5 - DNA, 29 bp, synthetic construct
  source 1..29: mol_type = other DNA; note = Primer F2
caggtgcagctgcaggagtctgggggagr

SEQ ID NO: 6 - DNA, 34 bp, synthetic construct
  source 1..34: mol_type = other DNA; note = Primer R2
ctagtgcggccgctgaggagacggtgacctgggt

SEQ ID NO: 7 - DNA, 20 bp, synthetic construct
  source 1..20: mol_type = other DNA; note = Primer F4
aatacgcaaaccgcctctcc

SEQ ID NO: 8 - protein, 108 aa, synthetic construct
  source 1..108: note = Nanobody Nb2
ESGGGLVQPGGSLRLSCAASGFTFSRYWMHWVRQAPGKGLEWVSAIITGSSSTYYADSVKGRFSISRDNAKGTMYLQMNSLKSEDTAVYYCTNLLDYWGQGIQVTVSS

SEQ ID NO: 9 - protein, 123 aa, synthetic construct
  source 1..123: note = Nanobody Nb3
ESGGGLVQTGDSLRLSCEASTRAFGDYTVGWFRQAPGKERKFVASINWDAAGIAYADSVKGRFTISRDNAKNTLYLQMNSLKPEDTGIYYCAAALRPYSYSLYRREEADFGSWGQGTQVTVSS

SEQ ID NO: 10 - protein, 117 aa, synthetic construct
  source 1..117: note = Nanobody Nb4
ESGGGLVQPGGSLRLSCAASGSIFSINAMGWYRQAPGKQRELVAAITSEGSTKYADSVKGRFTISRDNAKNTGYLQMNSLKPEDTAVYYCNALVIATISPQYNYDYWGQGTQVTVSS

(19) United States
(12) Patent Application Publication
(10) Pub. No.: US 2025/0155432 A1
(43) Pub. Date: May 15, 2025

(54) NANOBODY CLASSICAL SWINE FEVER VIRUS (CSFV)-E0-Nb1 AGAINST CSFV E0 PROTEIN, GENE ENCODING THE SAME AND USE THEREOF

(71) Applicants: QINGDAO AGRICULTURAL UNIVERSITY, Qingdao (CN); INNOVUS SOLAREX BIOTECH CO., LTD., Qingdao (CN)

(72) Inventors: Zhi CAO, Qingdao (CN); Dehua YIN, Qingdao (CN); Wenling LI, Qingdao (CN); Lihong ZHANG, Qingdao (CN); Siyu LI, Qingdao (CN); Yixin ZHANG, Qingdao (CN); Xiaoduo WANG, Qingdao (CN); Shuo LI, Qingdao (CN)

(21) Appl. No.: 18/884,570

(22) Filed: Sep. 13, 2024

(30) Foreign Application Priority Data
Nov. 9, 2023 (CN) 202311490452.8

(51) Int. Cl. G01N 33/543; C07K 16/10; G01N 33/569; G01N 33/68

(52) U.S. Cl. CPC G01N 33/54388; C07K 16/1081; G01N 33/56983; G01N 33/6857; C07K 2317/569; G01N 2333/183

(57) ABSTRACT

A nanobody classical swine fever virus (CSFV)-E0-Nb1 against a CSFV E0 protein, and an encoding gene and use thereof are provided, belonging to the technical field of biological detection. The nanobody CSFV-E0-Nb1 has an amino acid sequence shown in SEQ ID NO: 1 and can be expressed using an expression system. The nanobody is coupled with a quantum dot to obtain an immunochromatographic test strip for distinguishing antibodies against a CSFV E2 subunit vaccine strain from those of a wild strain infected on site, and there is a simple production process of the test strip. The immunochromatographic test strip can differentiate and diagnose the antibodies against the CSFV E2 subunit vaccine strain and the wild strain, and has the advantages of rapid, convenient, and instant detection, thus providing a new method for the detection of classical swine fever (CSF) purification.

### CROSS REFERENCE TO RELATED APPLICATION

This patent application claims the benefit and priority of Chinese Patent Application No. 202311490452.8 filed with the China National Intellectual Property Administration on Nov. 9, 2023, the disclosure of which is incorporated by reference herein in its entirety as part of the present application.

### REFERENCE TO SEQUENCE LISTING

A computer readable XML file entitled “GWP20240705465_seqlist”, that was created on Aug. 20, 2024, with a file size of about 12,188 bytes, contains the sequence listing for this application, has been filed with this application, and is hereby incorporated by reference in its entirety.

### TECHNICAL FIELD

The present disclosure belongs to the technical field of biological detection, and specifically relates to a nanobody classical swine fever virus (CSFV)-E0-Nb1 against CSFV E0 protein, and a gene encoding the same and use thereof.

### BACKGROUND

Classical swine fever (CSF) is a highly-contagious and fatal infectious disease of pigs caused by classical swine fever virus (CSFV). It significantly impacts animal health and the pig farming industry. CSF is a mandatory reportable disease stipulated by the World Organization for Animal Health (WOAH). CSFV has spread throughout the world, causing serious losses in the pig industry and having a certain impact on the economies of various countries. Although some countries have managed to eradicate CSF through vaccines and other means, CSF is still prevalent in many countries and regions around the world, including China. CSF infections have been re-reported in some countries or regions that are previously declared CSF-free. Established diagnostic methods for CSF, such as virus isolation, fluorescent antibody detection, antigen capture antibody enzyme-linked immunosorbent assay (ELISA), reverse transcription polymerase chain reaction, virus neutralization test, and antibody ELISA, have been widely used. However, the differential diagnosis between CSFV E2 subunit vaccine strain and wild strain still requires multiple or various detection methods, which complicates large-scale monitoring and field application in pig farms.

Typical nanobodies have affinities (equilibrium dissociation constants) in the nanomolar to picomolar range, making them well suited for use in ligand binding assays. Low production cost: a small size of nanobodies allows for easier production and high yields in moderate volumes of bacterial culture. Easily editable properties to suit application demands (i.e., to improve specificity and affinity and expand detection possibilities): genes encoding the nanobodies can be easily re-engineered to select for altered binding properties or epitope tagging. Robustness and long shelf life: compared to Igs and ScFv fragments, the nanobodies have strong thermal stability and can therefore be easily transported at most ambient temperatures. Targeting cryptic antigenic determinants: the small size of nanobodies allows them to enter cryptic antigen-binding sites. Low immunogenicity and rapid blood clearance: the small size of nanobodies allows them to be freely filtered in the glomerulus, facilitating excretion. Nanobodies have been used in a variety of antigen-antibody detections and demonstrate excellent properties as detection probe antibodies. However, there are few reports on the use of the nanobodies in combination with quantum dots. The stability of nanobodies can ensure the stability of quantitative detection.

Quantum dots have a series of unique and excellent optical properties, including: i) quantum dots are more stable than organic fluorescent molecules and are not prone to photobleaching; ii) they have a wide fluorescence absorption spectrum, capable of absorbing both single-photon and two-photon; iii) the emission spectrum is narrow (generally with a half-peak width of less than 40 nm) and symmetrical, with no long-wavelength tailing; iv) quantum dots exhibit high fluorescence intensity and high quantum yield (>20%), along with desirable biocompatibility; v) they exhibit unique quantum effect and a large Stokes shift; vi) compared with ordinary organic dyes, quantum dots have a longer fluorescence lifetime (20 ns to 40 ns), which is beneficial to improve the contrast and accuracy of detection results.

In summary, there is currently a lack of a rapid and convenient detection method for distinguishing CSFV E2 subunit vaccine strains from wild strains. Traditional immunochromatographic techniques such as colloidal gold have low detection sensitivity. Traditional ELISA detection methods require laboratory-related instruments and operations, while nucleic acid detection methods such as reverse-transcript polymerase chain reaction (RT-PCR) cannot evaluate the effectiveness of vaccine-induced immunity.

### SUMMARY

An objective of the present disclosure is to provide a nanobody CSFV-E0-Nb1 against CSFV E0 protein. Based on the nanobody and quantum dot immunochromatography, an immunochromatographic test strip is developed for antibodies against a CSFV E2 subunit vaccine strain and a wild strain. The test strip allows for timely, efficient, and rapid on-site distinguishment of infections caused by the CSFV E2 subunit vaccine strain and the wild strain. Additionally, the production process is straightforward, reducing production costs, and demonstrating significant potential for market application.

The present disclosure provides a nanobody CSFV-E0-Nb1 against CSFV E0 protein, where the nanobody CSFV-E0-Nb1 has the amino acid sequence of SEQ ID NO: 1.

The present disclosure further provides a nucleotide molecule encoding the nanobody CSFV-E0-Nb1.

In some embodiments, the nucleotide molecule has the nucleotide sequence of SEQ ID NO: 2.

The present disclosure further provides a method for expressing the nanobody CSFV-E0-Nb1, including: constructing a recombinant expression vector by ligating the nucleotide molecule to an expression vector, transforming the recombinant expression vector into a host cell to allow induced expression, and subjecting a resulting expressed product to purification to obtain the nanobody CSFV-E0-Nb1.

In some embodiments, the expression vector includes a prokaryotic expression vector.

The present disclosure further provides an immunochromatographic test strip for antibodies against a CSFV E2 subunit vaccine strain and a wild strain, including a gold-labeled pad, a test line, and a control line; where
the gold-labeled pad is coated with a quantum dot-coupled CSFV E0 protein and a quantum dot-coupled CSFV E2 protein; and
the quantum dot-coupled CSFV E0 protein and the quantum dot-coupled CSFV E2 protein are used for a test line T1 and a test line T2, respectively, and the nanobody CSFV-E0-Nb1 and a CSFV E2 protein-specific nanobody are used for a control line C1 and a control line C2, respectively.

In some embodiments, a preparation process of a quantum dot-coupled protein includes: incubating a water-soluble quantum dot with a 1-(3-dimethylaminopropyl)-3-ethylcarbodiimide hydrochloride (EDC·HCl) solution and an N-hydroxysuccinimide (NHS) solution in the dark; incubating a resulting first incubated product with β-mercaptoethanol and a protein; and subjecting a resulting second incubated product to blocking, centrifuging a resulting blocked product, and re-dissolving a resulting precipitate to obtain the quantum dot-coupled protein.

In some embodiments, the protein and the quantum dot are coupled at a pH value of 6 to 8;
the quantum dot and an activator are at a mass ratio of 1:2 to 1:8; and
the protein and the quantum dot are coupled at a mass ratio of 1:1 to 1:15.

In some embodiments, the test line is labeled with 50 μg/line to 150 μg/line of the protein; and
the control line is labeled with 75 μg/line to 175 μg/line of the nanobody.

In some embodiments, when the test line T1 is labeled with the quantum dot-coupled CSFV E0 protein, the test line T2 is labeled with the quantum dot-coupled CSFV E2 protein, the control line C1 is labeled with the CSFV-E0-Nb1 nanobody, and the control line C2 is labeled with the CSFV E2 protein-specific nanobody;
if there is no band on each of the test line T1, the test line T2, the control line C1, and the control line C2, it is determined that detection is unsuccessful and needs to be repeated;
if there is one or no band on the control line C1 or the control line C2, it is determined that the detection is unsuccessful and needs to be repeated;
if there is a band on each of the test line T1, the control line C1, and the control line C2 but there is no band on the test line T2, it is determined that the detection is unsuccessful and needs to be repeated;
if there is a band on each of the test line T1, the test line T2, the control line C1, and the control line C2, it is determined that there is an antibody produced by the wild strain;
if there is a band on each of the test line T2, the control line C1, and the control line C2 but there is no band on the test line T1, it is determined that there is an antibody produced by the CSFV E2 subunit vaccine strain; and
if there is a band on each of the control line C1 and the control line C2 but there is no band on each of the test line T1 and the test line T2, it is determined that a detection result is negative and no CSFV antibody is produced.

Beneficial effects: in the present disclosure, a CSFV E0-specific nanobody CSFV-E0-Nb1 is selected by using a phage display library technology of the CSFV E0 nanobody, and the CSFV-E0-Nb1 can be expressed using an expression system. In addition, the nanobody is coupled with the quantum dot to prepare an immunochromatographic test strips that can timely, efficiently, and quickly distinguish antibodies against the CSFV E2 subunit vaccine strain from those of the wild strain on site. The nanobody may reduce the cost of obtaining control antibodies and improve the stability of the test strip. High stability is conductive to quantitative detection, and vaccine immune effects can be evaluated without ELISA. Compared with traditional monoclonal antibodies, the nanobody is simpler to obtain and smaller in size, and shows better sensitivity and specificity. The application of quantum dot directly improves the sensitivity and specificity of immunochromatographic test strip. Compared with traditional colloidal gold test strips, the test strip of the present disclosure exhibits higher sensitivity and is more conducive to early detection of infection. The immunochromatographic test strip has a simple production process, reduces production costs, and shows significant potential for market application.

In the present disclosure, the immunochromatographic test strip may differentiate and diagnose the antibodies against the CSFV E2 subunit vaccine strain and the wild strain, and has the advantages of rapid, convenient, and instant detection. This provides a new method for the detection of classical swine fever (CSF) purification, which is simpler and faster than traditional two immunochromatographic test strips that adopt CSFV E2 and CSFV E0 proteins, respectively. The immunochromatographic test strip based on the nanobody and quantum dot may distinguish the infections caused by CSFV E2 subunit vaccine strain from those caused by wild strain on site conveniently, efficiently, and quickly at a low cost, thus providing a new detection method for the CSF purification.

### BRIEF DESCRIPTION OF THE DRAWINGS

FIG. 1 shows a schematic diagram of the test strip, where 1 is a sample pad, 2 is a label pad, 3 is a chromatography pad, 4 is a plastic bottom shell, and 5 is a nitrocellulose (NC) membrane;

FIG. 2 shows a binding diagram of each component when the test strip detects antibodies against the CSFV E2 subunit vaccine strain;

FIG. 3 shows a binding diagram of each component when detecting infection of the CSFV wild strain;

FIG. 4 shows titer determination of the CSFV E0 antibody in alpaca serum;

FIG. 5 shows a result of the first round of PCR amplification;

FIG. 6 shows a result of the second round of PCR amplification;

FIG. 7 shows an identification result of the positive rate after electroporation;

FIG. 8 shows results of the enrichment determination during solid phase panning;

FIG. 9 shows results of positive clones determined by indirect ELISA;

FIG. 10 shows analysis results of the monoclonal sequencing of four nanobodies, Nb1-Nb4 (SEQ ID NOS: 1 and 8-10);

FIG. 11 shows results of binding assays of four nanobodies;

FIG. 12 shows SDS-PAGE verification results of the expressed CSFV-E0-Nb1 nanobody, where lanes 1, 3, and 5 represent the precipitate after fragmentation, and lanes 2, 4, and 6 represent the supernatant after fragmentation;

FIG. 13 shows SDS-PAGE verification results of CSFV-E0-Nb1 nanobody after purification, where lane 1 represents the flow-through, and lanes 2 to 9 represent the eluate; and

FIG. 14 shows specificity validation results of the CSFV-E0-Nb1 nanobody.

### DETAILED DESCRIPTION OF THE EMBODIMENTS

The present disclosure provides a nanobody CSFV-E0-Nb1 against CSFV E0 protein, where the nanobody CSFV-E0-Nb1 has the amino acid sequence of SEQ ID NO: 1:

ESGGGLVQPGGSLR L S C A G S G I I F S S V
T M A W Y R Q A P G K Q R E V V A R F S
S G G R A T Y A D S V E G R F T I S R D
N V K N M V Y L Q M N S LA P E D T A V
Y Y C N A N W W Y E R N YD Y W G Q G T
Q V T V S S.

In the present disclosure, a process for constructing the nanobody CSFV-E0-Nb1 preferably utilizes a phage display library. The process more preferably includes: constructing a CSFV E0 nanobody phage display library, immunizing an alpaca, isolating lymphocytes, amplifying a target fragment, constructing a recombinant vector, and transforming the recombinant vector into a TG1 competent cell; screening a CSFV E0-specific nanobody, and then conducting solid phase panning three times, monoclonal crude expression and identification, sequencing analysis, and binding force determination in sequence. The process most preferably includes: immunizing the alpaca with the expressed CSFV E0 protein, collecting the lymphocytes, extracting a total RNA, amplifying a nanobody fragment, and ligating the fragment with a phage display vector pCANTAB-5E, transforming a resulting recombinant vector into the host TG1 competent cell, and then constructing a primary phage display library; rescuing the primary library with an M13KO7 phage to obtain a CSFV-specific nanobody phage display library, obtaining a positive monoclonal strain after three rounds of solid phase panning and indirect ELISA detection in sequence, and sequencing the positive monoclonal strain to obtain a sequence of the CSFV E0 protein-specific nanobody.

The present disclosure further provides a nucleotide molecule encoding the nanobody CSFV-E0-Nb1.

In the present disclosure, the nucleotide molecule preferably has the nucleotide sequence of SEQ ID NO: 2:

GAGTCTGGGGGAGGCTTGGTGCAGCCTGGGGGGTCTCTGAGACTCT
CCTGTGCAGGCTCAGGAATAATCTTCAGTAGCGTTACCATGGCCTG
GTACCGCCAGGCTCCAGGGAAGCAGCGCGAAGTGGTCGCTCGTTTT
AGTAGTGGTGGTCGCGCGACCTACGCAGACTCCGTGGAGGGCCGAT
TCACCATCTCCAGAGACAACGTCAAGAATATGGTCTATCTACAAAT
GAACAGCCTGGCACCTGAGGACACGGCCGTCTATTACTGTAATGCG
AACTGGTGGTACGAGAGGAATTATGATTACTGGGGCCAGGGGACCC
AGGTCACCGTCTCCTCAGC.

The present disclosure further provides a method for expressing the nanobody CSFV-E0-Nb1, including: constructing a recombinant expression vector by ligating the nucleotide molecule to an expression vector, transforming the recombinant expression vector into a host cell to allow induced expression, and subjecting a resulting expressed product to purification to obtain the nanobody CSFV-E0-Nb1.

In the present disclosure, the expression vector preferably includes a prokaryotic expression vector. In the examples, pET-28a is used as an example for illustration, but it cannot be regarded as the entire protection scope of the present disclosure. In some embodiments, the sequence set forth in SEQ ID NO: 2 is ligated to the pET-28a vector to construct a recombinant expression vector, which is then transformed into BL21 (DE3) competent cells to allow induced expression, and a resulting protein is purified to obtain the nanobody CSFV-E0-Nb1 of the present disclosure.

The present disclosure further provides an immunochromatographic test strip for antibodies against a CSFV E2 subunit vaccine strain and a wild strain, including a gold-labeled pad, a test line, and a control line; where
the gold-labeled pad is coated with a quantum dot-coupled CSFV E0 protein and a quantum dot-coupled CSFV E2 protein; and
the quantum dot-coupled CSFV E0 protein and the quantum dot-coupled CSFV E2 protein are used for a test line T1 and a test line T2, respectively, and the nanobody CSFV-E0-Nb1 and a CSFV E2 protein-specific nanobody are used for a control line C1 and a control line C2, respectively.

In the present disclosure, a preparation process of a quantum dot-coupled protein preferably includes: incubating a water-soluble quantum dot (CdSe) with an EDC. HCl solution and an NHS solution in the dark; incubating a resulting first incubated product with β-mercaptoethanol and a protein; and subjecting a resulting second incubated product to blocking, centrifuging a resulting blocked product, and re-dissolving a resulting precipitate to obtain the quantum dot-coupled protein. A marker of the immunochromatographic test strip is the water-soluble quantum dot, and the coupling of the protein and the water-soluble quantum dot is preferably conducted at a pH of 6-8, a mass ratio of the quantum dot to activator of 1:2 to 1:8, and a coupling amount of the protein to quantum dot of 1:1 to 1:15 μg; the coupling is more preferably conducted at a pH of 7, a mass ratio of the quantum dot to activator of 1:4, and a coupling amount of the protein to quantum dot of 1:15 μg.

In the present disclosure, the test lines of the immunochromatographic test strip are CSFV E0 and E2 proteins. For example, the test line T1 is for the CSFV E0 protein, and the test line T2 is for the CSFV E2 protein, with the labeling amount being preferably (50-150) μg, more preferably 100 μg.

In the present disclosure, the control lines of the immunochromatographic test strip correspond to CSFV E0 and E2 nanobodies. For example, the control line T1 is for the CSFV-E0-Nb1 nanobody, and the control line T2 is for the CSFV-E2-Nb1 nanobody, with the labeling amount of preferably (75-175) μg, more preferably 125 μg. The sequence and preparation method of CSFV-E2-Nb1 have been disclosed in Chinese patent CN114957454A and will not be described in detail here.

In the present, sensitivity, and repeatability of the immunochromatographic test strip are comparable to or stronger than those of commercial test strips. When the test line T1 is labeled with the quantum dot-coupled CSFV E0 protein, the test line T2 is labeled with the quantum dot-coupled CSFV E2 protein, the control line C1 is labeled with the CSFV-E0-Nb1 nanobody, and the control line C2 is labeled with the CSFV E2 protein-specific nanobody;
if there is no band on each of the test line T1, the test line T2, the control line C1, and the control line C2, it is determined that detection is unsuccessful and needs to be repeated;
if there is one or no band on the control line C1 or the control line C2, it is determined that the detection is unsuccessful and needs to be repeated;
if there is a band on each of the test line T1, the control line C1, and the control line C2 but there is no band on the test line T2, it is determined that the detection is unsuccessful and needs to be repeated;
if there is a band on each of the test line T1, the test line T2, the control line C1, and the control line C2, it is determined that there is an antibody produced by the wild strain;
if there is a band on each of the test line T2, the control line C1, and the control line C2 but there is no band on the test line T1, it is determined that there is an antibody produced by the CSFV E2 subunit vaccine strain; and
if there is a band on each of the control line C1 and the control line C2 but there is no band on each of the test line T1 and the test line T2, it is determined that a detection result is negative and no CSFV antibody is produced.

In the present disclosure, the immunochromatographic test strip is scanned with a gold-labeled immunoassay analyzer, the ratios of T1/C1 and T2/C2 are read, and then a standard curve is established, such that the CSFV antibody can be quantitatively detected.

In order to further illustrate the present disclosure, the nanobody CSFV-E0-Nb1 of a CSFV E0 protein, the gene encoding the same and the use thereof provided by the present disclosure are described in detail below with reference to the accompanying drawings and examples, but the accompanying drawings and the examples should not be construed as limiting the protection scope of the present disclosure.

The experimental methods in the following examples that are not specified with specific conditions are generally conducted under conventional conditions, for example, conditions disclosed in Molecular Cloning: Experiment Guide (Sambrook et al., New York: Cold Spring Harbor Laboratory Press, 1989) or conditions recommended by manufacturers.

### Example 1

Construction of the primary phage display library of the nanobody against CSFV E0 protein

### 1.1 Immunization of Alpacas

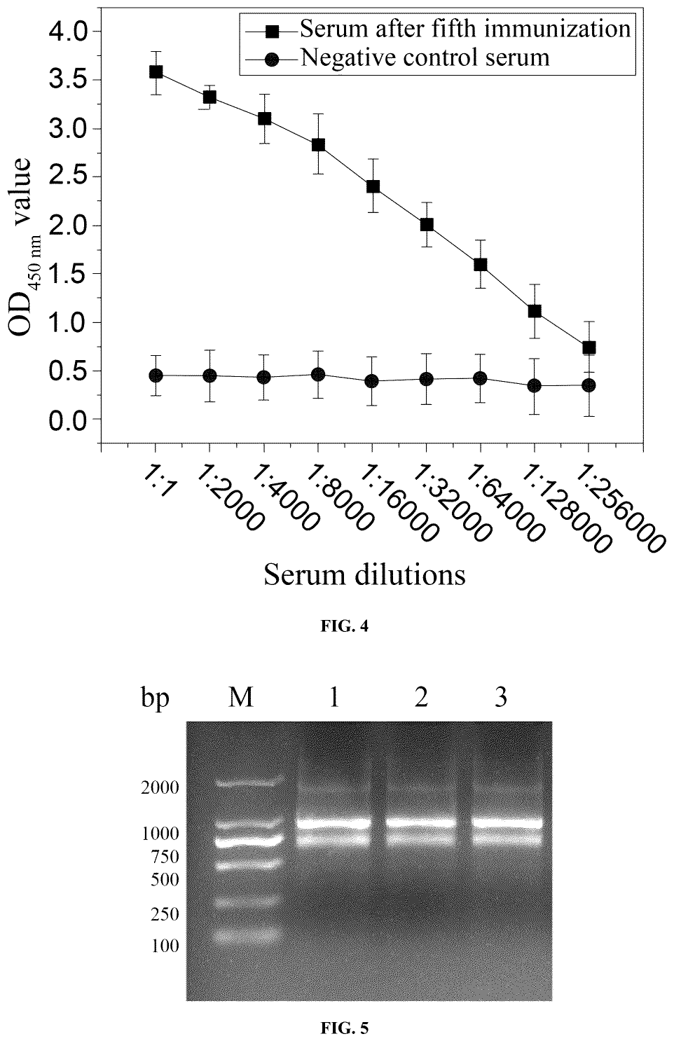

Adult male healthy alpacas were immunized with 5 mg of CSFV E0 protein (GenBank: KY816734.1) each time for a total of 5 times, with an interval of 2 weeks. After each immunization, serum was collected and a CSFV E0 antibody titer was determined by indirect ELISA. When the serum antibody titer was ≥10⁵, it was considered to meet the library construction requirements. The results are shown in FIG. 4. After the fifth immunization, the serum antibody titer reached 1.28×10⁵, indicating that there was a desirable immunization effect. Twenty milliliters of anticoagulated blood was then collected from the carotid artery for isolation of total lymphocytes.

### 1.2 Isolation of Total Lymphocytes

In order to obtain the nanobody sequence, the total lymphocytes were isolated from the collected anticoagulated blood, and the serum, plasma, red blood cells, and total lymphocytes were isolated by gradient centrifugation. The collected total lymphocytes were washed twice, and then the extracted total lymphocytes were counted.

### 1.3 Nanobody Fragment Amplification

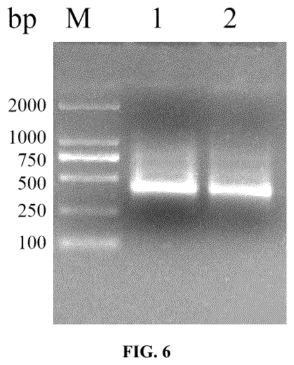

Total RNA was extracted from the total lymphocytes. During this process, care should be taken to prevent contamination in order to increase the yield of total RNA. The extracted total RNA was immediately reverse transcribed into cDNA for easy storage and later use. Two pairs of primers were designed based on the conserved sequences of alpaca-specific nanobodies, making it easier to obtain nanobody target sequences with higher purity and larger quantities. The first pair of primers was used to amplify the conserved regions of the heavy and light chains of traditional IgG, as well as the heavy and conserved regions of the nanobody. Gel electrophoresis analysis showed that there were target bands at 700 bp and 900 bp, and the target band of the nanobody at 700 bp was recovered (FIG. 5). The second pair of primers was used to amplify the heavy chain of the nanobody using the gel recovery product as a template, and the primers had Pst I and Not I restriction sites, respectively. Gel electrophoresis analysis showed that there was a nanobody gene band at 400 bp, and this part was recovered (FIG. 6).

### First Pair of Primers:

F1 (SEQ ID NO: 3):
GTCCTGGCTGCTCTTCTACAAGG;
R1 (SEQ ID NO: 4):
GGTACGTGCTGTTGAACTGTTCC;

### Second Pair of Primers:

F2 (SEQ ID NO: 5):
CAGGTGCAGCTGCAGGAGTCTGGGGGAGR;
R2 (SEQ ID NO: 6):
CTAGTGCGGCCGCTGAGGAGACGGTGACCTGGGT.

### 1.3 Construction of Phage Display Vector for Nanobody

The phage display vector pCANTAB 5E and the gel-recovered product were double-digested with Pst I and Not I restriction endonucleases, respectively, and then the digested products were purified. The digested products were ligated using T4 ligase at a molar ratio of 1:7 between the vector and the target gene at 4° C. overnight, and then a ligated and incubated product was purified using a purification kit and the concentration was measured.

### 1.4 Preparation of TG1 Electroporation Competent Cells

A TG1 bacterial solution and LB broth were inoculated into a conical flask at a volume ratio of 1:50 and cultured at 37° C. until the OD₆₀₀ value reached 0.5. A collected bacterial solution was incubated on ice for 30 min and centrifuged at 4,500 rpm for 10 min. A supernatant was discarded, the pelleted bacterial cells were resuspended with an equal volume of 10% glycerol, and centrifuged again. A resulting supernatant was discarded, the pelleted bacterial cells were resuspended with ½ volume of 10% glycerol, and centrifuged again. A supernatant was discarded, the pelleted bacterial cells were resuspended with ¼ volume of 10% glycerol, and centrifuged again. A resulting supernatant was discarded, and the pelleted bacterial cells were gently resuspended with an appropriate amount of 10% glycerol to allow electroporation.

1.5 the Ligated and Purified Product Electroporated into TG1 Competent Cells

The purified ligation product was mixed with the prepared TG1 electroporation competent cells, and the electroporation instrument parameters were set as follows: C=25 μF, PC=200Ω, V=1.8 kV. After electroporation, all products were placed at 37° C. and incubated for 60 min. The incubated products were then spread on a plate medium and the library was measured. After 12 h, the library was collected, the bacterial cells were collected with a cell scraper, and then added into an equal volume of 60% glycerol for preservation.

### 1.6 Characterization of Phage Display Library

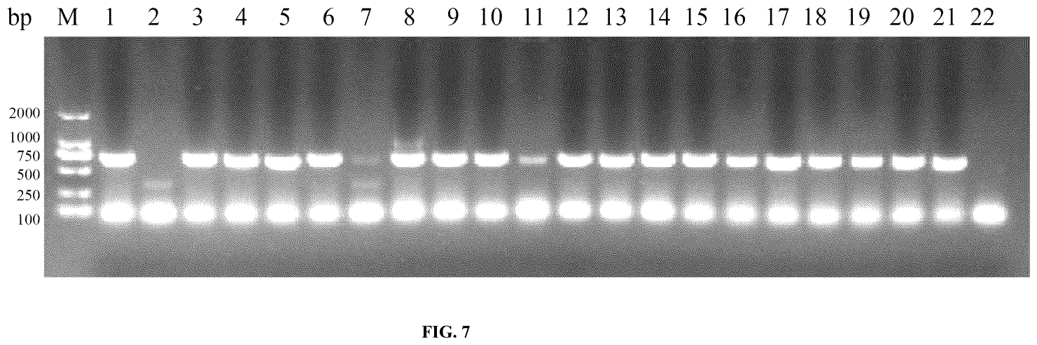

Twenty one monoclones were randomly selected from the plate for library identification, and identified by colony PCR (identification primers were F4 and R2), and the library capacity positive rate was calculated. The results (FIG. 7) showed that a total of 21 monoclones were selected (lane 22 as a negative control), of which 2 were negative and 19 were positive, such that the library capacity positive rate was 90.48%.

The primer F4 (SEQ ID No. 7): AATACGCAAACCGCCTCTCC, was located about 335 bp upstream of the multiple cloning restriction site of the pCANTAB 5E vector.

### Example 2

### Panning of CSFV E0-Specific Nanobody

### 2.1 Rescue of Phage Display Library

The constructed phage display library was rescued using helper phage M13KO7. The constructed phage display library was inoculated into a medium and cultured until the OD₆₀₀ value reached 0.6. The M13KO7 phage at 20 MOI was calculated and added. After incubation for 30 min, a resulting product was centrifuged at 3,000 rpm for 10 min, a resulting supernatant was discarded, and a resulting bacterial pellet was resuspended in the medium, and cultured overnight. At this time, the M13KO7 phage might specifically recognize and infect the TG1 bacteria, and adsorb the phage display vector pCANTAB-5E to the tail end of the phage to form a recombinant phage. The pellet was concentrated using PEG6000/NaCl solution. Specifically: the bacterial solution was centrifuged at 6,000 rpm for 15 min, a resulting supernatant was collected, an equal volume of PEG6000/NaCl solution was added, and incubated on ice for 4 h; after the incubation was completed, a product was centrifuged, a supernatant was discarded, and the pellet was resuspended with an appropriate amount of phosphate-buffered saline (PBS); a product was incubated at 4° C. for 12 h, centrifuged, and a supernatant was collected to obtain the phage display library of CSFV E0 nanobody.

### 2.2 Method for Determination of Phage Titer

The phage was diluted 10-fold to 10⁻¹² with broth or PBS solution. 100 μL of the 10⁻⁶, 10⁻⁸, 10⁻¹⁰, and 10⁻¹² dilutions were taken, respectively, and added into an equal volume of TG1 bacterial solution in the logarithmic growth phase and allowed to infect for 30 min. The incubated product was spread on plate medium and cultured overnight, the number of colonies on each plate was counted, and the phage titer was calculated.

The titer of recombinant phages from the phage display library after rescue was calculated according to this method.

### 2.3 Solid Phase Panning of CSFV E0-Specific Nanobody

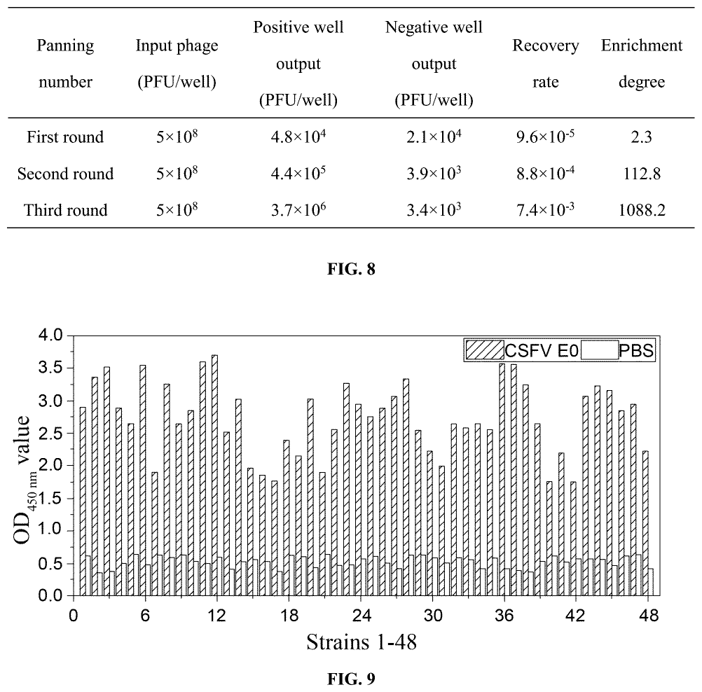

The CSFV E0 protein was coated on the ELISA plate at 10 μg per well; after the coating incubation, a resulting product was blocked with 2.5% skim milk powder for 2 h; after the blocking and washing, a resulting recombinant phage was incubated for 2 h; after the incubation, a resulting product was washed 2 times, and the recombinant phage bound to CSFV E0 was eluted with freshly-prepared 0.1 M triethylamine (TEA), and an equal volume of 1 M Tris-HCl was quickly added to neutralize the TEA. The eluted recombinant phage was titered, and the ratio of the wells coated with CSFV E0 protein to the wells not coated with protein was calculated; if it did not reach 10³ or above, the eluted recombinant phage was amplified; after the amplification, the solid phase panning was repeated. It was generally believed that the solid phase panning was completed when the ratio reached 10³ or above; the results (FIG. 8) showed that an enrichment degree was 1088 in the third round of panning, indicating that there was a desirable enrichment effect.

### 2.4 Crude Expression of Recombinant Phage Monoclone

In order to better identify the recombinant phage of CSFV E0-specific nanobody, 48 single colonies on the plate during the last recombinant phage titer determination were randomly selected for separate culture, and tryptone-broth (TB) and isopropyl β-D-1-thiogalactopyranoside (IPTG) were added to allow induced expression of the recombinant nanobody vector on the recombinant phage. When the monoclonal clone was cultured to OD₆₀₀ of 0.6, an IPTG solution with a final concentration of 1 mM was added to allow induced expression overnight; the bacterial pellets were centrifuged separately, and were repeatedly frozen and thawed at −80° C. for 3 times. After the last resuspension with PBS, the bacterial pellet was centrifuged at 6,000 rpm for 10 min, and a resulting supernatant was collected to obtain the crude extract of the recombinant nanobody.

### 2.5 Indirect ELISA Identification

To identify CSFV E0-specific nanobody, crude expressed monoclonal antibodies were measured by indirect ELISA using a principle of antigen-antibody specific binding. Four micrograms of CSFV E0 protein was coated on each well of the ELISA plate. After the coating, a resulting product was blocked with 2.5% skim milk powder for 2 h. The crude extract was incubated, and 1 parallel well of each crude extract group was incubated with PBS solution as a negative control. Rabbit anti-E-Tag monoclonal antibody was used as a primary antibody. Horseradish peroxide (HRP)-labeled goat anti-rabbit IgG antibody was used as a secondary antibody. After 3,3′,5,5′-Tetramethylbenzidine (TMB) colorimetric solution was added, the colorimetric reaction was terminated with a stop solution, and the OD₄₅₀ value was measured with an ELISA reader. When the crude extract well was greater than 3 times the value of the negative control well, it was considered positive, otherwise it was negative. The results (FIG. 9) showed that all 48 selected monoclonal clones were positive.

### 2.6 Positive Monoclonal Sequencing Analysis

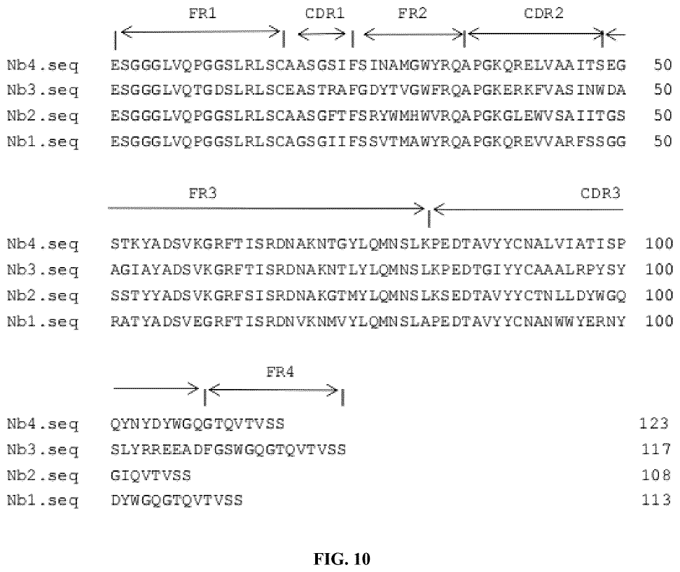

The bacterial suspension that was positive by indirect ELISA was sent to Sangon for sequencing. The sequencing results were subjected to amino acid alignment for analysis. The results are shown in FIG. 10, and a total of 4 nanobodies with different amino acid sequences were aligned.

### 2.7 Verification of the Binding Capacity of CSFV E0 Protein Nanobody

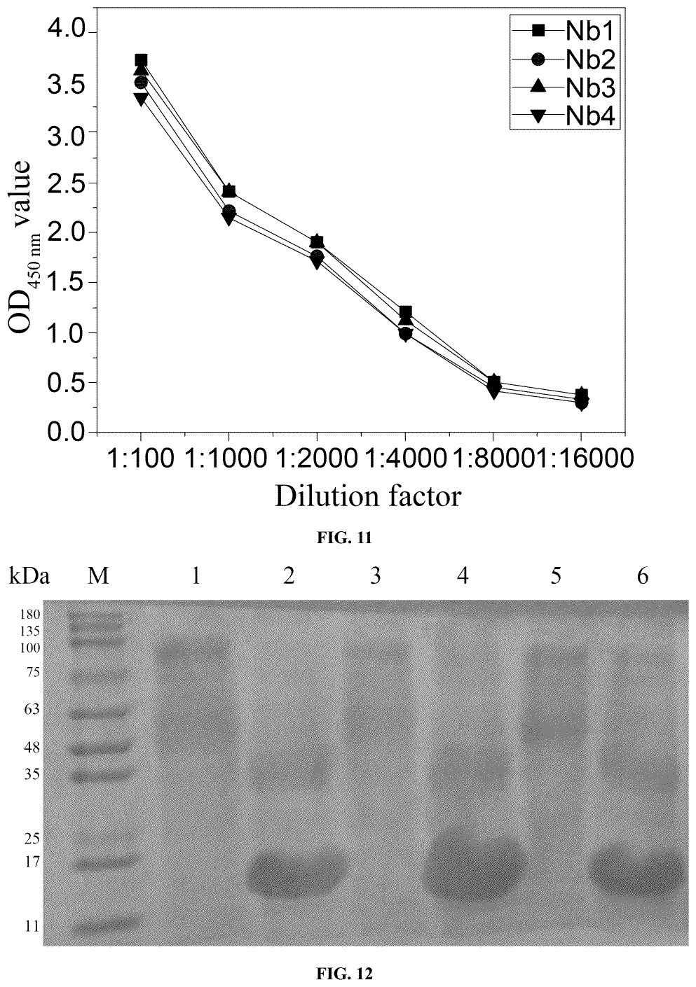

After amino acid alignment of the sequencing results, the binding capacity of different nanobodies was verified. The crude extracts of different nanobodies were diluted to the same concentration, and the remaining steps were the same as those for indirect ELISA to determine positive clones. The results are shown in FIG. 11. All the 4 nanobodies exhibited high binding capacity, among which nanobody 1 had the highest binding capacity (named CSFV-E0-Nb1).

### Example 3

### Expression of CSFV E0-Specific Nanobody

### 3.1 Construction of CSFV-E0-Nb1 Expression Vector

The sequenced Nb1 sequence was subjected to codon usage optimization for expression in the pET-28a vector (SEQ ID NO: 2), and the pET-28a-Nb1 recombinant expression plasmid was synthesized by a biological company.

3.2 Induced Expression of pET-28a-Nb1

The recombinant expression plasmid synthesized by the biological company was transformed into the expression competent cells of Escherichia coli BL21 (DE3). After incubation, a resulting product was spread on the plate medium. After overnight culture, 3 single clones were selected and inoculated into the broth. When the bacterial solution was cultured to OD₆₀₀ of 0.6, IPTG with a final concentration of 1 mM was added to allow induced expression. After 4 h of the induced expression, centrifugation was conducted at 6,000 rpm for 15 min, and a resulting pellet was resuspended in PBS. The bacterial cells were lysed by ultrasonic crusher, followed by centrifugation to collect the supernatant and the pellet separately. The protein expression and solubility were verified by SDS-PAGE. The results (FIG. 12) showed that all 3 strains were soluble, among which the 2nd strain had the highest expression level.

### 3.3 Purification of CSFV-E0-Nb1 Protein

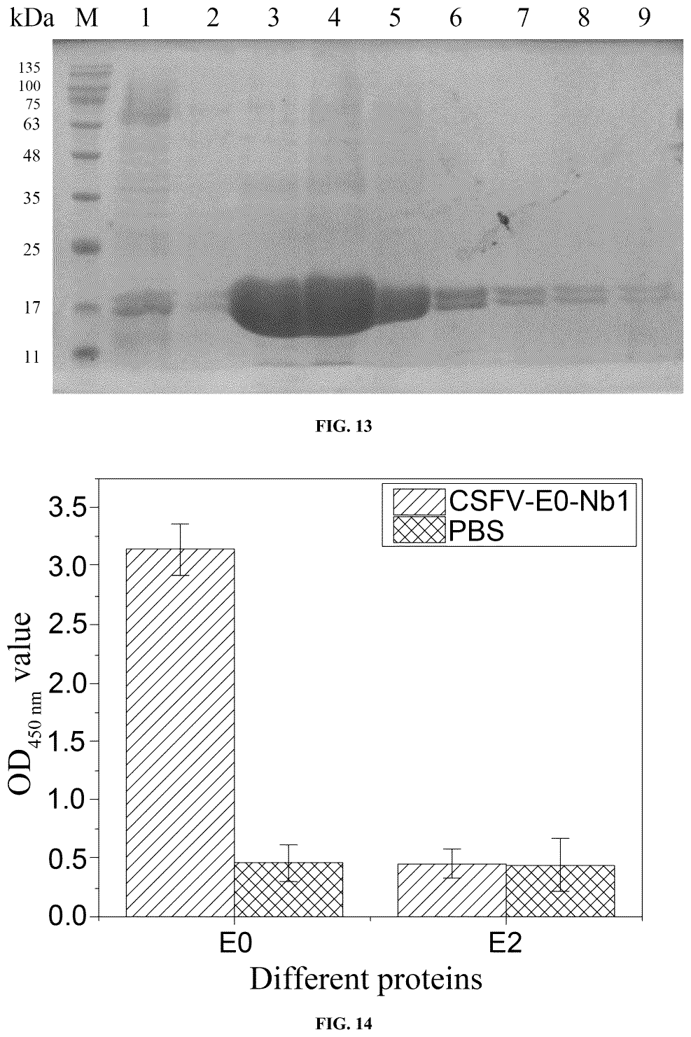

Ni-NTA column was used for purification following the experimental steps set forth in the kit instructions; after purification, the protein was subjected to SDS-PAGE to verify its purity. The results (FIG. 13) showed that there was a desirable purification effect without other impurity protein bands.

### 3.4 Specificity Verification of CSFV-E0-Nb1 Protein

The specificity of the expressed protein was verified by indirect ELISA. The CSFV E2 and E0 proteins were coated on the ELISA plate in equal amounts, blocked with 2.5% skim milk powder for 2 h, incubated with CSFV-E0-Nb1 protein after washing; after incubation, the proteins were incubated with rabbit anti-His tag antibody, and then with HRP-labeled goat anti-rabbit IgG antibody; after color development with TMB and termination with stop solution, the OD₄₅₀ value was read by an ELISA instrument. The results (FIG. 14) showed that CSFV-E0-Nb1 did not bind to CSFV E2, but specifically bound to CSFV E0 protein.

### Example 4

### Establishment of an Immunochromatographic Differential Diagnosis Method for Quantum Dot

### 4.1 Quantum Dot-Coupled CSFV E0 and E2 Proteins

An appropriate amount of water-soluble quantum dots (CdSe) was added into EDC·HCl solution and NHS solution, and incubated for 2 h in the dark; after the incubation, β-mercaptoethanol was added; the CSFV E0 and E2 proteins were added and incubated for 12 h; after the incubation, 7.5% glycine (Gly) solution+1% BSA solution was added to allow blocking for 2 h; a resulting product was centrifuged at 12,000 rpm for 15 min, a supernatant was discarded, and the pellet was re-dissolved to obtain the quantum dot-coupled CSFV E0 and E2 proteins.

### 4.2 Screening of Quantum Dot Coupling Conditions

Screening for optimal pH value: pH values were set to 6, 7, 8, 9, and 10 to observe whether protein aggregation or precipitation occurred in the coupling. The results showed that as the pH value increased, both CSFV E0 and E2 proteins were coupled and aggregated or precipitated, that is, the optimal pH value was 7.

Selection of the amount of activator: the activator EDC has a function of carboxylating the —COOH on the surface of quantum dots, thereby promoting coupling with the target protein. At the optimal pH value, the mass ratio of quantum dots to EDC was diluted from 1:1 to 1:16, and the coupling were observed. The results showed that when the mass ratio of EDC of the quantum dots was 1:4, no aggregation would occur. In order to reduce costs, the amount of activator was set to 4 times that of the quantum dots.

Screening of the optimal coating amounts for CSFV E0 and E2 proteins: the E0 and E2 proteins each were diluted to 1 mg/mL; under the conditions of optimal pH and activator dosage, the proteins were divided into 5 groups, with 5 μL, 10 μL, 15 μL, 20 μL, and 25 μL added to each group in sequence. The results showed that with a smaller amount added, the aggregation and precipitation of the coupling product could occur, and the fluorescence intensity could increase with the increase of the amount added. However, there was little difference between the three groups of 15 μL, 20 μL, and 25 μL, that is, an optimal amount of protein added was 15 μg.

### 4.3 Preparation of Test Line and Screening of Optimal Concentration

The CSFV E0 and E2 proteins were diluted to 1 mg/mL, and then diluted again at 1:5, 1:10, 1:20, 1:40, and 1:80, which were marked on the NC membrane; the positive serum and negative serum were tested in sequence, and the results at different concentrations were observed. The results showed that the fluorescence intensity gradually weakened with the increase of dilution, while the fluorescence intensity was similar at 1:5 and 1:10, that is, 1:10 was selected as an optimal concentration.

### 4.4 Preparation of Control Line and Screening of Optimal Concentration

The CSFV E2 protein nanobody used in the control line was the nanobody in patent CN114957454A, and the CSFV E0 protein nanobody was selected and prepared in the present disclosure. The purified CSFV-E0-Nb1 and CSFV-E2-Nb1 were diluted to 1 mg/mL, subsequently diluted in a series of 1:2, 1:4, 1:8, and 1:16 ratios, which were marked on the NC membrane; PBS buffer, positive serum, and negative serum were tested in sequence to observe the results at different concentrations. The results showed that as the dilution ratio increased, the fluorescence intensity gradually decreased, but the intensity was too strong at 1:2 and more moderate at 1:8. Hence, 1:8 was selected as an optimal concentration.

### 4.5 Assembly of Differential Diagnosis Test Strip

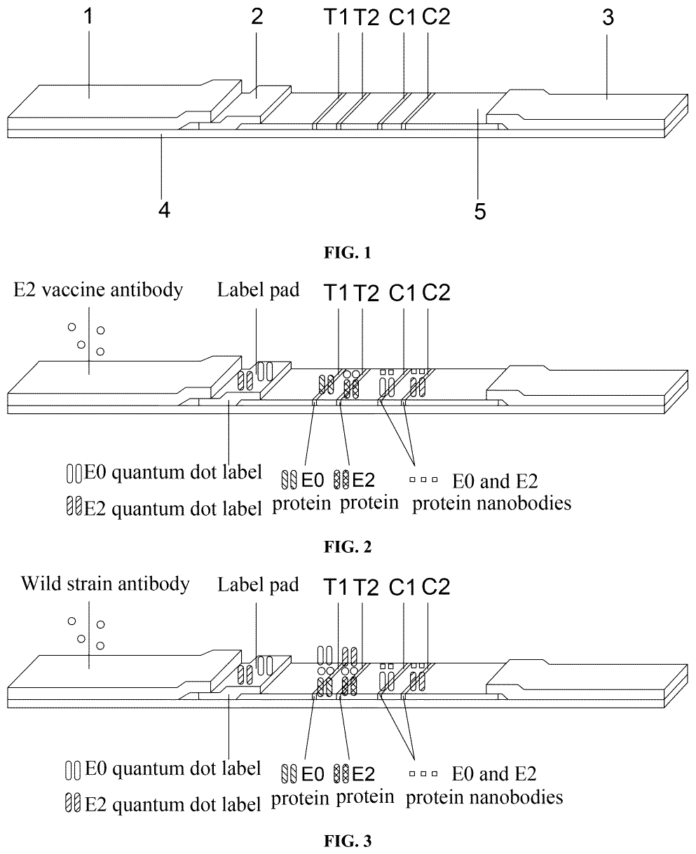

The gold-labeled pad was sprayed with quantum dots to label CSFV E0 and E2 proteins, with a dosage of 15 μg each; test line T1 was labeled with CSFV E0 protein, with a labeling amount of 100 μg; test line T2 was labeled with CSFV E2 protein, with a labeling amount of 100 μg; control line C1 was labeled with CSFV-E0-Nb1 protein, with a labeling amount of 125 μg; control line C2 was labeled with CSFV-E2-Nb1 protein, with a labeling amount of 125 μg; while other conditions were all optimal conditions. The assembly diagram of the test strip is shown in FIG. 1.

### 4.6 Result Determination Standards

If there was no band on each of the T1, the T2, the C1, and the C2, it was determined that detection was unsuccessful and needed to be repeated; if there was one or no band on the C1 or the C2, it was determined that the detection was unsuccessful and needed to be repeated; if there was a band on each of the T1, the C1, and the C2 but there was no band on the T2, it was determined that the detection was unsuccessful and needs to be repeated; if there was a band on each of the T1, the T2, the C1, and the C2, it was determined that there was an antibody produced by the wild strain (FIG. 3); if there was a band on each of the T2, the C1, and the C2 but there was no band on the T1, it was determined that there was an antibody produced by the CSFV E2 subunit vaccine strain (FIG. 2); and if there was a band on each of the C1 and the C2 but there was no band on each of the T1 and the T2, it was determined that a detection result was negative and no CSFV antibody was produced.

### 4.7 Specificity Verification

The prepared test strips were used to detect the serum collected after immunization with CSFV attenuated vaccine, the serum collected after immunization with CSFV E2 subunit vaccine, the standard CSFV negative serum, the standard porcine pseudorabies virus-positive serum, and the standard porcine parvovirus-positive serum. The serum prepared by immunization with the CSFV attenuated strain vaccine had T1 and T2 with color development; the serum prepared by immunization with the CSFV E2 subunit vaccine had T1 without color development and T2 with color development; the standard CSFV negative serum, the standard porcine pseudorabies virus positive serum, and the standard porcine parvovirus positive serum had T1 and T2 without color development. The above results showed that the established immunochromatographic detection method could diagnose and distinguish CSFV E2 subunit vaccine from wild strain, and had desirable specificity.

### 4.8 Sensitivity Verification

The same serum sample collected after immunization with CSFV attenuated vaccine and the serum collected after immunization with CSFV E2 subunit vaccine were diluted five-fold to 1:3125; the prepared immunochromatographic test strip and a purchased commercial CSFV antibody colloidal gold test strip were used for testing. The prepared immunochromatographic test strip could still accurately identify the infection type at 1:625, while the commercial colloidal gold test strip had no longer clear results at 1:25. The above results showed that the established immunochromatographic test strip had higher sensitivity and accuracy.

### 4.9 Repeatability Verification

The immunochromatographic test strip prepared in different batches showed the same test results for the same serum. The immunochromatographic test strip prepared in the same batch was tested by different operators on the same serum, and the results were also the same. This indicated that there were desirable inter-batch and intra-batch repeatability of the prepared immunochromatographic test strip.

In summary, the immunochromatographic test strip based on quantum dot and nanobody prepared in the present disclosure may stably and highly sensitively differentiate between CSFV E2 subunit vaccine strain and wild strain infections, and can provide a convenient, low-cost, rapid, and efficient detection method for CSFV detection in CSF clearance. It is also worth noting that the immunochromatographic test strip is scanned with a gold-labeled immunoassay analyzer, and ratios of T1/C1 and T2/C2 are read to establish a standard curve, such that the CSFV antibody can be quantitatively detected, which is conductive to the evaluation of vaccine efficacy and antibody titer.

Although the above examples have described the present disclosure in detail, they are only a part of, not all of, the embodiments of the present disclosure. Other embodiments may also be obtained by persons based on the examples without creative efforts, and all of these embodiments shall fall within the protection scope of the present disclosure.

What is claimed is:

1. A nanobody classical swine fever virus (CSFV)-E0-Nb1 against CSFV E0 protein, wherein the nanobody CSFV-E0-Nb1 has the amino acid sequence of SEQ ID NO: 1.

2. A nucleotide molecule encoding the nanobody CSFV-E0-Nb1 according to claim 1.

3. The nucleotide molecule according to claim 2, wherein the nucleotide molecule has the nucleotide sequence of SEQ ID NO: 2.

4. A method for expressing the nanobody CSFV-E0-Nb1 according to claim 1, comprising: constructing a recombinant expression vector by ligating a nucleotide molecule encoding the nanobody CSFV-E0-Nb1 to an expression vector, transforming the recombinant expression vector into a host cell to allow induced expression, and subjecting a resulting expressed product to purification to obtain the nanobody CSFV-E0-Nb1.

5. The method according to claim 4, wherein the nucleotide molecule has the nucleotide sequence of SEQ ID NO: 2.

6. The method according to claim 4, wherein the expression vector comprises a prokaryotic expression vector.

7. An immunochromatographic test strip for antibodies against a CSFV E2 subunit vaccine strain and a wild strain, comprising a gold-labeled pad, a test line, and a control line; wherein
the gold-labeled pad is coated with a quantum dot-coupled CSFV E0 protein and a quantum dot-coupled CSFV E2 protein; and
the quantum dot-coupled CSFV E0 protein and the quantum dot-coupled CSFV E2 protein are used for a test line T1 and a test line T2, respectively, and the nanobody CSFV-E0-Nb1 according to claim 1 and a CSFV E2 protein-specific nanobody are used for a control line C1 and a control line C2, respectively.

8. The immunochromatographic test strip according to claim 7, wherein a preparation process of a quantum dot-coupled protein comprises: incubating a water-soluble quantum dot with a 1-(3-dimethylaminopropyl)-3-ethylcarbodiimide hydrochloride (EDC·HCl) solution and an N-hydroxysuccinimide (NHS) solution in the dark; incubating a resulting first incubated product with β-mercaptoethanol and a protein; and subjecting a resulting second incubated product to blocking, centrifuging a resulting blocked product, and re-dissolving a resulting precipitate to obtain the quantum dot-coupled protein.

9. The immunochromatographic test strip according to claim 8, wherein the protein and the quantum dot are coupled at a pH value of 6 to 8;
the quantum dot and an activator are at a mass ratio of 1:2 to 1:8; and
the protein and the quantum dot are coupled at a mass ratio of 1:1 to 1:15.

10. The immunochromatographic test strip according to claim 7, wherein the test line is labeled with 50 μg/line to 150 μg/line of the protein; and
the control line is labeled with 75 μg/line to 175 μg/line of the nanobody.

11. The immunochromatographic test strip according to claim 7, wherein when the test line T1 is labeled with the quantum dot-coupled CSFV E0 protein, the test line T2 is labeled with the quantum dot-coupled CSFV E2 protein, the control line C1 is labeled with the CSFV-E0-Nb1 nanobody, and the control line C2 is labeled with the CSFV E2 protein-specific nanobody;
if there is no band on each of the test line T1, the test line T2, the control line C1, and the control line C2, it is determined that detection is unsuccessful and needs to be repeated;
if there is one or no band on the control line C1 or the control line C2, it is determined that the detection is unsuccessful and needs to be repeated;
if there is a band on each of the test line T1, the control line C1, and the control line C2 but there is no band on the test line T2, it is determined that the detection is unsuccessful and needs to be repeated;
if there is a band on each of the test line T1, the test line T2, the control line C1, and the control line C2, it is determined that there is an antibody produced by the wild strain;
if there is a band on each of the test line T2, the control line C1, and the control line C2 but there is no band on the test line T1, it is determined that there is an antibody produced by the CSFV E2 subunit vaccine strain; and
if there is a band on each of the control line C1 and the control line C2 but there is no band on each of the test line T1 and the test line T2, it is determined that a detection result is negative and no CSFV antibody is produced.
